(19) United States
(12) Patent Application Publication
(10) Pub. No.: US 2003/0012749 A1
(43) Pub. Date: Jan. 16, 2003

(54) Preparations for the non-traumatic excision of a nail

(76) Inventors: Karl Kraemer, Langen (DE); Manfred Bohn, Hofheim (DE)

Correspondence Address: Klaus Schweitzer, ProPat, Crosby Building, 2912 Crosby Road, Charlotte, NC, 28211-2815, US

(21) Appl. No.: 10/149,577

(22) PCT Filed: Dec. 12, 2000
(86) PCT No.: PCT/EP00/12553

(30) Foreign Application Priority Data
Jan. 3, 2000 (DE) 100 00 053.3

(51) Int. Cl. A61K 7/04

(52) U.S. Cl. 424/61

(57) ABSTRACT

This invention relates to a preparation containing preferably a hydrophilic film-former, water and urea. Said preparation contains urea in a quantity of between 70 and 90 % by weight and the hydrophilic film-former in a quantity of between 10 and 30 % by weight, the respective quantities being measured in relation to the non-volatile constituents. The preparations are suitable for the non-traumatic excision of diseased toe and finger-nails, for example of fungus-infested areas of toe and finger-nails.

### BACKGROUND OF THE INVENTION

Fungal diseases of the toenails or fingernails (onychomycoses) are a widespread pathological condition. Their treatment is recognized as being difficult and lengthy. According to estimates published in the specialist medical literature up to 8% of the population have a nail mycosis and in the age group from 40 to 60 years it is even up to 20%.

Contrary to the opinion expressed to date, nail mycoses are far more than just a cosmetic problem. Whereas diseased toenails may, inter alia, have unpleasant adverse effects on movement, affected fingernails often lead to an adverse effect on self confidence and the quality of life. A fungally infected nail may further act as a reservoir of pathogens and cause infections on other areas of the body.

There are currently various ways of treating fungal diseases of nails:

One method of treatment, the systemic one, consists of oral administration of antifungal agents. Experience has shown that this may on occasion lead to serious unwanted side effects of the pharmaceutical, which may be life-threatening in some circumstances, because the active ingredient must reach the focus of infection via the blood circulation.

A newer method is much more advantageous from the toxicology viewpoint and consists of treating the affected nails topically with special antimycotic-containing nail preparations, in particular with lacquer preparations (U.S. Pat. No. 4,957,730; 5,264,206). In contrast to the systemic therapy, unwanted side effects or interactions with other systemically administered medicinal substances are virtually precluded on topical treatment.

An additional local procedure which is practised successfully to reduce the treatment time both of systemic and of local treatment of onychomycoses is removal of the affected areas of the nail. Thus, for example, it is recommended to remove as much as possible of the damaged nail material with a nail file or scissors before starting the actual treatment. Total extraction of the nails, in particular on multiple infection, is in most cases unacceptable for the patients and involves the risk of irreversible damage to the nail matrix with the consequence of the growth of deformed nails.

Both methods thus have serious disadvantages. Whereas it is true that the filing can be carried out by the patients themselves, though this makes a large contribution to the spread of the pathogens, the cutting with scissors or a scalpel must for safety reasons be undertaken by an experienced skilled person. In a large group of problem patients such as diabetics in particular, injuries may be followed by infections which, in the worse case, may lead to amputations of limbs. This is, however, a great disadvantage from the ethical and from the pharmaco-economic viewpoints.

In order to minimize the risk associated therewith, another method has therefore become used in therapy for the treatment of fungus-infected areas of nails on the hands and feet to detach the nail. This method is based on the keratolytic effect of highly concentrated urea (40%) or of potassium iodide or sodium iodide (50%) on the diseased nail substance. Healthy nail material is not attacked thereby.

Thus, for example, urea-containing or KI- or NaI-containing anhydrous pastes are applied to the diseased nails until the entire surface of the nails has a thin covering. The affected fingernails and toenails are then covered with a plaster or a dressing for 24 hours in each case. The plaster is then detached and the affected fingers or toes are bathed in warm water for about 10 minutes. The treatment must be continued until the softened diseased nail substance can be completely removed with a scraper. This generally takes from 7 days to 14 days, depending on the extent of the disease and thickness of the nails. It is recommended that zinc paste be applied to protect the areas of skin surrounding the nail.

This method has also failed to achieve conclusive success because patients often do not persist with the treatment for cosmetic reasons and for reasons of time—for example because of the inconvenient and unsightly plasters or dressings on the toes and fingers and because the procedures are necessary each day. A further disadvantage is regarded as being the unpleasant odor which is perceptible on removal of the plaster dressings.

The purpose of the invention is to provide a formulation which does not have the described disadvantages in removal of diseased nail substance from the toenails or fingernails.

### BRIEF DESCRIPTIONS OF THE INVENTION

It has now been found that pathologically altered areas of nails on fingers and toes, which may occur, for example, as a result of a fungal, bacterial or viral infection, or, for example, as a result of psoriasis, can be detached simply and efficiently if the preparations according to the invention are applied to the diseased nails. These preparations are non-solid and preferably non-pasty. The preferably liquid preparations according to the invention can, because of their properties, be applied easily and accurately like a nail varnish with a brush and, after they have dried, are resistant to wiping and rubbing off. It is moreover unnecessary to cover the preparations with plaster dressings or apply a special protective film for the areas of skin surrounding the nail, or bathe the affected areas, e.g. fingers and toes, each day. In addition, no unpleasant odor is produced during the treatment.

### DETAILED DESCRIPTION OF THE INVENTION

The invention therefore relates to cosmetic and/or pharmaceutical preparations preferably comprising a hydrophilic film former, water and urea.

The invention further relates to the use of the preparations according to the invention for atraumatic nail removal, i.e. for the removal of the pathologically altered nail substance. Healthy nail material is not attached thereby.

The preparations according to the invention preferably comprise urea in an amount from 70 to 90 percent by weight, preferably 75 to 85 percent by weight, based on the involatile ingredients of the preparations. The purpose according to the invention, the detachment of infected areas, such as, for example, fingernails and toenails, or of areas of skin which may be keratinized, can also be achieved with other urea concentrations. The present invention likewise encompasses corresponding preparations. Altered concentrations may have an effect on the treatment duration.

Example 6 represents a particularly preferred embodiment.

Examples of suitable hydrophilic film formers are acrylic/methacrylic ester copolymers, polyvinylpyrrolidones, polyvinyl alcohols, vinyl acetate/vinylpyrrolidone copolymers, vinyl acetate/crotonic acid copolymers, methyl vinyl ether/maleic acid copolymers, carboxymethylcellulose, hydroxyethylcellulose, hydroxypropylcellulose and hydroxypropylmethylcellulose, Polyvinylpyrrolidones are particularly suitable. They are employed in amounts of from 10 to 30 percent by weight, preferably 15 to 25 percent by weight, based on the involatile ingredients.

Further aids which are suitable are plasticizers such as glycerol triacetate or 1.2-propylene glycol, and agents for adjusting the pH of the preparations, for example lactic acid or citric acid. The pH-regulating substances are preferably employed in amounts of from 0.5 to 5 percent by weight, based on the finished preparation. The amount of plasticizers is preferably 1 to 10 percent by weight, based on the finished preparation.

The preparations according to the invention may also comprise additives customary in cosmetics, such as, for example, phthalate-glyceryl triacetate- or camphor-based plasticizers, dyes or colored pigments, pearlescent agents, sedimentation retarders, sulfonamide resins, silicates, fragrances, wetting agents such as, for example, sodium dioctyl sulfosuccinate, lanolin derivatives, light stabilizers such as 2-hydroxy-4-methoxybenzophenone or substances with antibacterial activity. Colored or pigmented nail lacquers have the advantage, for example, that the preparation according to the invention can be adapted to the esthetic sense of the patient and the existing nail changes are not directly visible to other people. The preparations may also comprise substances with antimycotic activity, such as, for example, hydroxypyridones such as ciclopirox, ciclopirox olamine, piroctone or rilopirox, morpholine derivatives such as amorolfine hydrochloride or amorolfine, azoles such as bifonazole, butoconazole, fluconazole, clotrimazole, econazole, itraconazole, miconazole, omoconazole, oxiconazole, croconazole, fenticonazole, sulconazole, tioconazole, terconazole, ketoconazole or isoconazole or allyl compounds such as terbinafine hydrochloride, terbinafine or naftifine, and griseofulvin, haloprogin, mepartricin, undecylenic acid, dodecyltriphenylphosphonium hydrochloride, lauroyloxypropylaminobutyric acid, tolciclate, tolnaftate and butenafine.

Further examples of suitable hydroxypyridones which may be mentioned are: 1-hydroxy-4-methyl-6-n-hexyl-, -6-isohexyl-, -6-n-heptyl- or -6-isoheptyl -2-pyridone, 1-hydroxy-4-methyl-6-octyl- or -6-isooctyl-2-pyridone, in particular 1-hydroxy-4-methyl-6-(2,4,4-trimethylpentyl)-2-pyridone, 1-hydroxy-4-methyl -6-cyclohexyl-2-pyridone, 1-hydroxy-4-methyl-6-cyclohexylmethyl- or -6-cyclo-hexylethyl -2-pyridone, it also being possible in each case for the cyclohexyl radical to carry a methyl radical, 1-hydroxy-4-methyl-6-(2-bicyclo[2,2, 1]heptyl) -2-pyridone, 1-hydroxy-3,4-dimethyl-6-benzyl- or -6-dimethylbenzyl-2-pyridone and 1-hydroxy-4-methyl-6-(β-phenylethyl)-2-pyridone.

Said active ingredients can also be used in the form of their salts or adducts, e.g. as olamines, which are suitable for pharmacological and/or cosmetic purposes.

The present invention also encompasses preparations which comprise alkali metal iodides, preferably KI or NaI, in place of or in combination with urea. It is also possible to use mixtures of the alkali metal iodides. The concentrations (% by weight) of the alkali metal iodides or of the urea/alkali metal iodide combinations preferably correspond to those described for the urea-containing preparations. Water as volatile ingredient may be replaced wholly or partly by other substances (solvents). The precondition is that the urea and the alkali metal iodides are sufficiently soluble in the substance (liquid) used or in the substance mixture (liquid mixture) used. Examples which can be used are water/alcohol mixtures. The complete or partial replacement of water may advantageously lead for example to a faster formation (drying) of the urea-containing film on the treated areas.

The preparations according to the invention are suitable not only for atraumatic removal of pathologically altered nail substance but also in general for the removal of pathologically altered keratinous areas such as, for example, keratinized areas of skin which may arise, for example, as a result of fungal, bacterial or viral infection or, for example, as a result of psoriasis. Such a removal of pathologically altered keratinous areas may be used to prepare for maximally efficient subsequent treatment of a person. The preparations are produced in a manner known per se by combination and mixing of the individual components and—where necessary—further processing appropriate for the particular preparation.

The present invention is explained in detail by the following examples, but not restricted thereto. Unless otherwise noted, the stated amounts (% by weight) are based on weight of the finished preparation (including the volatile ingredients; e.g. water).

### EXAMPLE 1

A preparation according to the invention has the following composition:

| | |
|---|---|
| Urea | 40.0% |
| Polyvinylpyrrolidone (molecular weight about 11 500) | 10.0% |
| Demineralized water | 50.0% |

### EXAMPLE 2

A preparation according to the invention has the following composition:

| | |
|---|---|
| Urea | 40.0% |
| Polyvinylpyrrolidone (molecular weight about 11 500) | 10.0% |
| Glycerol triacetate | 4.0% |
| Lactic acid | 1.0% |
| Demineralized water | 45.0% |

### EXAMPLE 3

A preparation according to the invention has the following composition:

| | |
|---|---|
| Urea | 40.0% |
| Vinyl acetate/vinylpyrrolidone copolymer | 7.5% |
| Glycerol triacetate | 2.5% |
| Demineralized water | 50.0% |

### EXAMPLE 4

A preparation according to the invention has the following composition:

| | |
|---|---|
| Urea | 40.0% |
| Polyvinyl alcohol | 8.0% |
| 1.2-Propylene glycol | 2.0% |
| Demineralized water | 50.0% |

### EXAMPLE 5

A preparation according to the invention has the following composition:

| | |
|---|---|
| Urea | 40.0% |
| Polyvinylpyrrolidone (molecular weight about 25 000) | 10.0% |
| Fluconazole | 2.5% |
| Lactic acid | 1.0% |
| Demineralized water | 46.5% |

### EXAMPLE 6

A preparation according to the invention has the following composition:

| | |
|---|---|
| Urea | 40% |
| Polyvinylpyrrolidone | 10% |
| Lactic acid | 1% |
| Water | 49% |

### Comparative Example

A prior art paste preparation has the following composition:

| | |
|---|---|
| Urea | 40.0% |
| Anhydrous lanolin | 20.0% |
| Bleached wax | 5.0% |
| White petrolatum | 35.0% |

Activity test

Groups of 20 patients infected with a nail fungus were treated with the preparations according to the invention or with a prior art paste as in the comparative example.

The preparations according to the invention were applied with a brush directly to the affected nails once a day before retiring to bed. The urea-containing film which resulted on the nails about 2 minutes after application was resistant to wiping and water. It was therefore unnecessary to provide special protection for the areas of skin surrounding the nails or apply plaster dressings. Because of the high water content of the preparations, the affected fingernails or toenails were not additionally bathed.

The urea paste according to the comparative example was likewise applied once a day, covering the areas of skin surrounding the nails with a protective paste. The affected fingernails or toenails were covered with a plaster dressing for 24 hours in each case and bathed in warm water for about 10 minutes after removal of the dressing.

Result:

After treatment for about 7 days it was possible to remove the affected areas of nails and the subungual tissue detritis easily from both groups of patients. The treatment with the preparations according to the invention was, however, greatly preferred because of its simplified procedure—and the time-saving associated therewith for the patient.

1. A method for the removal and treatment of diseased Keratin areas such as finger or foot nails being affected by onychomycosis, comprising applying to such areas a preparation comprising water, urea and a film-forming agent.

2. The method of claim 1 for simultaneous treatment of nail mycosis.

3. The method of claim 1 or 2, wherein said preparation contains urea in an amount of about 70 to about 90 weight-% or the film-forming agent in an amount of about 10 to about 30 weight-%, or urea in an amount of about 70 to about 90 weight-% and the film-forming agent in an amount of about 10 to about 30 weight-%, based on the non-volatile constituents.

4. The method of claim 1 or 2, wherein the film-forming agent is polyvinylpyrrolidone.

5. The method of claim 1 or 2, wherein the preparation further comprises a plasticizer or pH-regulating agents or a plasticizer and pH-regulating agents.

6. The method of claim 1 or 2, wherein the preparation further comprises an antimycotically active substance.

7. The method of claim 6, wherein said antimycotically active substance is selected from a group comprising: hydroxypyridones, morpholine derivatives, azoles, or allyl compounds, and griseofulvin, haloprogin, mepartricin, undecylenic acid, dodecyltriphenylphosphonium hydrochloride, lauroyloxypropylaminobutyric acid, tolciclate, tolnaftate and butenafine or their salts or adducts suitable for pharmacological purposes.

8. The method of claim 7, wherein the hydroxypyridone is ciclopirox, ciclopiroxolamine, piroctone or rilopirox.

9. The method of claim 7, wherein the morpholine derivative is amorolfine hydrochloride or amorolfine.

10. The method of claim 7, wherein the azole is bifonazole, fluconazole, clotrimazole, econazole, itraconazole, miconazole, oxiconazole, croconazole, fenticonazole, tioconazole, ketoconazole or isoconazole.

11. The method of claim 7, wherein the allyl compound is terbinafine hydrochloride, terbinafine or naftifine.

12. The method of claim 6, wherein said antimycotically active substance is fluconazole.

13. The method of claim 1 or 2, wherein in said preparation urea is partially or completely replaced by alkali iodines.

14. The method of claim 1 or 2, wherein in said preparation water is partially or completely replaced by substances in which or in the mixture of substances with water urea or the alkali iodines or urea and the alkali iodines are soluble.

15. The method of claim 1 or 2, wherein said preparation is a liquid preparation.

16. The method of using a preparation of claim 1 in cosmetics, whtich method comprises the step of incorporating a preparation of claim 1 into a cosmetic.
